(19) United States
(12) Patent Application Publication
(10) Pub. No.: US 2009/0263340 A1
(43) Pub. Date: Oct. 22, 2009

(54) COSMETIC COMPOSITIONS

(75) Inventors: Claudia Ille-Boehler, Loerrach (DE); Jochen Klock, Freiburg (DE); Horst Westenfelder, Neustadt a.d.w. (DE)

Correspondence Address: NIXON & VANDERHYE, PC, 901 NORTH GLEBE ROAD, 11TH FLOOR, ARLINGTON, VA, 22203, US

(73) Assignee: DSM IP ASSETS B.V., Heerlen (NL)

(21) Appl. No.: 12/297,285

(22) PCT Filed: Apr. 3, 2007
(86) PCT No.: PCT/EP2007/002993

§ 371 Date: Oct. 15, 2008

(30) Foreign Application Priority Data
Apr. 18, 2006 (EP) 06007959.7

(51) Int. Cl. A61K 8/368; A61K 8/67; A61K 31/60; A61P 17/10; A61Q 19/02; A61Q 19/08

(52) U.S. Cl. 424/62; 514/161

(57) ABSTRACT

The present invention relates to cosmetic or dermatological compositions comprising salicylic acid, optionally substituted salicylic acid and/or derivatives thereof and ascorbic acid and/or derivatives thereof in a cosmetically acceptable carrier. The salicylic acid, optionally substituted salicylic acid and/or derivatives thereof serve as skin penetration enhancer for the ascorbic acid and/or derivatives thereof into human skin. The compositions are especially useful as skin lightening agents, anti-acne agents or anti-ageing agents.

The present invention relates to cosmetic or dermatological compositions comprising salicylic acid, optionally substituted salicylic acid and/or derivatives thereof and ascorbic acid and/or derivatives thereof in a cosmetically acceptable carrier. The salicylic acid, optionally substituted salicylic acid and/or derivatives thereof serve as skin penetration enhancer for the ascorbic acid and/or derivatives thereof into human skin. The compositions are especially useful as skin lightening agents, anti-acne agents or anti-ageing agents.

There is a growing demand for cosmetic or dermatological composition which can be effectively used for minimizing or even eliminating skin hyper-pigmentation and/or pigmentation disorders (age spots, freckles, blotches, darkening, uneven tone, and the like), wrinkling and other chronical changes typically associated with skin ageing or environmental damage to human skin. At the same time such cosmetic or dermatological compositions should preferably exhibit no skin irritation. However, it is well known that actives agents may cause skin irritations which are highly unwanted. Thus, identifying a composition that has improved efficacy relative to those described in the prior art having at the same time a low irritation potential would be of significant commercial interest.

Ascorbic acid and/or derivatives thereof are widely used in cosmetic or dermatological compositions, particularly in skin care preparations, for various purposes such as skin-lightening, removal of age spots, as anti-acne agent or as anti-ageing agent.

Salicylic acid is an aromatic acid used in cosmetic formulations e.g. as an exfolient, hair-conditioning agent, anti-dandruff or skin-conditioning agent. Salicylic acid derivatives are also known to be used in cosmetics e.g. as preservatives, UV-absorbers, fragrant ingredients or solvents.

Improved penetration of ascorbic acid and/or derivatives thereof would increase their performance as more of the active agent reaches the necessary site of action in the skin. As a consequence of the improved efficacy, this could also result in lower amounts of the active agents needed to achieve the desired effects thereof.

It has now surprisingly been found that salicylic acid, optionally substituted salicylic acid and/or derivatives thereof significantly enhance the penetration of ascorbic acid and/or derivatives thereof into human skin.

Thus, one aspect the present invention is related to cosmetic or dermatological compositions comprising
(a) 0.01 to 8 wt. %, preferably 0.5 to 5 wt. % of salicylic acid, optionally substituted salicylic acid and/or derivatives thereof;
(b) 0.01 to 20 wt. %, preferably 0.5 to 10 wt. % of ascorbic acid and/or derivatives thereof and
(c) a cosmetically acceptable carrier.

Preferably, the salicylic acid, optionally substituted salicylic acid and/or derivatives thereof are used as skin penetration enhancer.

In a more preferred embodiment the cosmetic or dermatological compositions comprise
(a) 0.01 to 8 wt. %, preferably 0.5 to 5 wt. % of salicylic acid, optionally substituted salicylic acid and/or derivatives thereof as skin penetration enhancer;
(b) 0.01 to 20 wt. %, preferably 0.5 to 10 wt. % of ascorbic acid and/or derivatives thereof and
(c) a cosmetically acceptable carrier,
   wherein the molar ratio of the salicylic acid, optionally substituted salicylic acid and/or derivatives thereof to the ascorbic acid and/or derivatives thereof is in the range of 0.1:5 to 5:0.1, preferably in the range of 0.5:2 to 2:0.5, most preferred in the range of about 0.7:1.2 to 1.2:0.7.

Preferred are cosmetic or dermatological compositions as defined above which are skin-lightening compositions, anti-ageing compositions or compositions for the prevention, reduction or treatment of acne.

The present invention also pertains to the use of cosmetic or dermatological compositions as defined above for the control of melanin formation, for enhancing the collagen deposition and for an efficient antioxidant protection. In particular, the invention relates to the use of cosmetic or dermatological compositions as defined above for skin-lightening and/or for the prevention, reduction or treatment of skin ageing and/or acne.

In another embodiment the present invention relates to a method of lightening the skin, a method of providing an anti-ageing benefit and/or a method of prevention, reduction or treatment of acne comprising the step of applying an effective amount of a cosmetic or dermatological composition as defined above to the skin of a subject in need of such a treatment.

The topical application is preferably applied at least once per day but can also be applied several times a day e.g. two or three times a day. Usually it takes at least two days until the desired effect is achieved. However, it can take several weeks or even months until the desired effect is achieved.

The amount of the cosmetic or dermatological composition which is to be applied to the skin depends on the concentration of the active ingredients in the compositions and the desired cosmetic or pharmaceutical effect. For example, application can be such that a crème is applied to the skin. A crème is usually applied in an amount of about 1 to 2 mg crème/cm² skin. The amount of the composition which is applied to the skin is, however, not critical, and if with a certain amount of applied composition the desired effect cannot be achieved, a higher concentration of the active ingredients can be used e.g. by applying more of the composition or by applying compositions which contain more active ingredient. A person skilled in the art can thus easily determine the effective amount of a cosmetic composition to be applied.

In another aspect, the present invention relates to the use of salicylic acid, optionally substituted salicylic acid and/or derivatives thereof in cosmetic or dermatological compositions for enhancing the penetration of ascorbic acid and/or derivatives thereof, into the human skin. The molar ratio of salicylic acid, optionally substituted salicylic acid and/or derivatives thereof to ascorbic acid and/or derivatives thereof may be in the range of 0.1:5 to 5:0.1, preferably in the range of from 0.5:2 to 2:0.5, most preferred in the range of 0.7:1.2 to 1.2:0.7. The amount of salicylic acid in compositions comprising an ascorbic acid and/or derivatives thereof is not critical. Preferably 0.01 to 8 wt. %, most preferred 0.1-5% wt. % based on the total weight of the composition are used.

In another aspect, the invention relates to a method of enhancing the efficacy of ascorbic acid and/or derivatives thereof which comprises adding to a composition containing an ascorbic acid and/or derivatives thereof an effective amount of salicylic acid, optionally substituted salicylic acid and/or derivatives thereof. The term “effective amount salicylic acid, optionally substituted salicylic acid and/or derivatives thereof” means generally at least a concentration of 0.01% by weight of the salicylic acid, optionally substituted salicylic acid and/or derivatives thereof based on the total weight of the composition. Preferably, a concentration of 0.01 to 8 wt. %, most preferred of 0.1 to 5 wt. % based on the total weight of the composition is used. The amount of ascorbic acid and/or derivatives thereof in the composition is not critical and may be easily chosen by a person skilled in the art. Preferably a concentration of 0.01 to 20 wt. %, most preferred of 0.5 to 10 wt. % based on the total weight of the composition is used. The molar ratio of salicylic acid, optionally substituted salicylic acid and/or derivatives thereof to ascorbic acid and/or derivatives thereof may be in the range of 0.1:5 to 5:0.1, preferably in the range of from 0.5:2 to 2:0.5, most preferred in the range of 0.7:1.2 to 1.2:0.7.

The ascorbic acid and/or derivatives thereof for use in accordance with the present invention may be ascorbic acid and/or any non-toxic, non skin-irritating water-soluble or oil-soluble ascorbic acid derivative. The term ascorbic acid and/or derivatives thereof encompasses ascorbic acid as well as esters of ascorbic acid, and ester salts of ascorbic acid such as ascorbyl phosphates as well as ascorbic acid derivatives such as ascorbyl palmitate, ascorbyl tetraisopalmitate (for example available from Nikko Chemical), ascorbyl dipalmitate (for example, NIKKOL CP available from Nikko Chemical), ascorbyl linoleate, ascorbyl octanoate, 2-O-D-glucopyranosyl-L-ascorbic acid, which is an ester of ascorbic acid and glucose and usually referred to as L-ascorbic acid 2-glucoside or ascorbyl glucoside, and its metal salts.

The term “ascorbyl phosphate” as used herein denotes metal salts of mono- and poly-phosphoric acid esters of ascorbic acid wherein the phosphorylated hydroxy group of the ascorbic acid molecule features one or more phosphoric acid (phosphate) units, and metal cations, e.g. sodium and/or magnesium or calcium ions, are also present. The term “poly” generally denotes 2-10, preferably 2-4, phosphate units. The ascorbyl phosphates may also be referred to in general as “ascorbyl (poly)phosphates” to embrace both mono- and polyphosphates. Typical ascorbyl phosphates for use in the present invention are L-ascorbic acid phosphate ester salts such as sodium ascorbyl phosphate, potassium ascorbyl phosphate, magnesium ascorbyl phosphate, calcium ascorbyl phosphate and sodium magnesium L-ascorbyl-2-monophosphate. The ascorbyl phosphates are essentially present in the form of a hydrate or a dehydrate, which are included herein. Commercially available ascorbyl phosphates comprise trisodium L-ascorbyl-2-monophosphate which is available as STAY-C®50 form DSM Nutritional Products AG, (4303 Kaiseraugst, Switzerland) and magnesium L-ascorbyl phosphate available from Showa Denko) and sodium magnesium L-ascorbyl-2-monophosphate. The preferred ascorbyl phosphate for the purposes of the present invention is trisodium L-ascorbyl-2-monophosphate, in particular in the form of a dihydrate.

In all above embodiments preferably an ascorbyl phosphate is used. Preferably sodium or sodium magnesium or sodium calcium ascorbyl phosphate or mixtures thereof, most preferably trisodium-L-ascorbyl-2-monophosphate is incorporated into the cosmetic or dermatological composition according to the invention.

The term ‘salicylic acid, optionally substituted salicylic acid and/or derivatives thereof’ as used herein encompasses the free acid as well as salts or derivatives thereof. Suitable salts which can be used according to the invention may be selected from sodium salicylate, potassium salicylate, magnesium salicylate, calcium salicylate or methanolamine (MEA, diethanolamine (DEA) or triethanolamine (TEA) salicylate without being limited thereto. Suitable salicylic acid derivatives which can be used in accordance with the invention are e.g. the esters thereof such as C1-C25 alkyl esters or an aryl ester. Examples of such esters are e.g. capryloyl salicylate, C12-15 Alkyl salicylate, isocetyl salicylate, isodecyl salicylate, tridecyl salicylate, butyloctyl salicylate, hexyldodecyl salicylate, ethylhexyl salicylate, methyl salicylate, myristyl salicylate The salicylic acid and/or derivatives thereof can optionally be substituted with suitable substituents, one or several, which may be selected independently from the group of alkyl, aryl, alcohol, ether, ester, cyanide, amide, amine, sulfate, phosphate, fluoro, chloro, bromo or iodo groups or carbonyl groups.

The salicylic acid, optionally substituted salicylic acid and/or derivatives thereof are preferably selected from salicylic acid, sodium salicylate, potassium salicylate or magnesium salicylate.

In accordance with the invention, the topical or dermatological compositions as described above may be used for treatment and prevention of pigmentation disorders, such as

primary hyperpigmentation disorders which include those that are nevoid, congenital or acquired such as local hyperpigmented disorders which include pigmented nevi, ephelides juvenile freckles, an inherited characteristic; age spots; and café-au-lait spots) and lentigines (solar lentigines, senile lentigines, senile freckles, liver-spots); and
secondary hyperpigmentation disorders which include those occurring after a separate dermatologic condition, including acne; such disorders are most commonly seen in dark-skinned individuals and are called post inflammatory hyperpigmentation.
further hyperpigmentation disorders which include arsenical melanosis and disorders associated with Addison's disease; freckling and café-au-lait spots produced by neurofibromatosis; regional or patterned hyperpigmentation caused by melanocytic hyperactivity, such as idiopathic melasma occurring either during pregnancy or secondary to estrogen-progesterone contraception.

Other examples for disorders which may be treated or prevented by cosmetic or dermatological compositions according to the invention include the pigmentation following physical trauma, eczematoid eruptions are lupus erythematosus, and dermatoses such as pityriasis rosea, psoriasis, dermatitis herpetiformis, fixed drug eruptions, photodermatitis and Lichen simplex chronicus, tinea versicolor (under specific environmental conditions for a yeast type of skin fungus, present on normal skin) and acanthosis nigricans; post-inflammatory hyperpigmentations which can result due to abrasion, burns, wounds, insect bites, dermatitis, and other similar small, fixed pigmented lesions; Berloque hyperpigmentation, which is due to phototoxicity from chemicals in the rinds of limes and other citrus fruits, and to celery; and accidental hyperpigmentation which can result from post-lesional photosensitization and scarring.

Further examples of pigmentation disorders include those caused by some drugs, including chloroquine, chlorpromazine, minocycline and amiodarone. Benzoyl peroxide, fluorouracil and tretinoin can cause hyperpigmentation; fixed drug eruptions can result from phenolphthalein in laxatives, trimethoprim-sulfamethoxazole, nonsteroidal anti-inflammatory drugs (NSAIDs) and tetracyclines.

In certain forms of leukoderma such as vitiligo where, if the injured skin cannot be repigmented, the residual zone of normal skin are depigmented to impart a homogeneous white color to the entire skin. In all these conditions treatment with compositions in accordance with the invention may be considered.

Examples of cosmetic or dermatological compositions comprising ascorbic acid and/or derivatives thereof and salicylic acid, optionally substituted salicylic acid and/or derivatives thereof according to the present invention are skin care preparations, in particular, body lotions, body creams, body foams, body gels, facial lotions, facial creams, facial gels, e.g. eye creams, anti-wrinkle creams, day care lotions, night creams, treatment creams, treatment solutions, skin protection ointments, sunscreens, moisturizing gels, moisturizing creams, moisturizing sprays, revitalizing body sprays, cellulite gels, anti acne preparations, cleansing milks and peeling preparations.

The cosmetic or dermatological compositions according to the invention have a pH in the range of 3-10, preferably in the range of pH of 4-8, most preferred in the range of pH 5.5-7.5.

The cosmetic or dermatological compositions according to the invention such as the skin care preparations can contain further adjuvants and additives such as preservatives/antioxidants, fatty substances/oils, water, organic solvents, silicones, thickeners, softeners, emulsifiers, light screening agents, antifoaming agents, moisturizers, fragrances, surfactants, fillers, sequestering agents, anionic, cationic, nonionic or amphoteric polymers or mixtures thereof, propellants, acidifying or basifying agents, dyes, colorants, pigments or nanopigments, active ingredients, preservatives, insect repellants, or any other ingredients usually formulated into cosmetics. The necessary amounts of the cosmetic and dermatological adjuvants and additives can, based on the desired product, easily be chosen by a skilled artisan in this field and will be illustrated in the examples, without being limited hereto.

### Light Screening Agents

Additional screening agents are advantageously selected from IR, UV-A, UV-B, UV-C and/or broadband filters. Examples of UV-B or broad spectrum screening agents, i.e. substances having absorption maximums between about 290 nm and 340 nm may be organic or inorganic compounds. Organic UV-B or broadband screening agents are e.g. acrylates such as 2-ethylhexyl 2-cyano-3,3-diphenylacrylate (octocrylene, PARSOL® 340), ethyl 2-cyano-3,3-diphenylacrylate and the like; camphor derivatives such as 4-methyl benzylidene camphor (PARSOL® 5000), 3-benzylidene camphor, camphor benzalkonium methosulfate, polyacrylamidomethyl benzylidene camphor, sulfo benzylidene camphor, sulphomethyl benzylidene camphor, therephthalidene dicamphor sulfonic acid and the like; Cinnamate derivatives such as ethylhexyl methoxycinnamate (PARSOL® MCX), ethoxyethyl methoxycinnamate, diethanolamine methoxycinnamate (PARSOL® Hydro), isoamyl methoxycinnamate and the like as well as cinnamic acid derivatives bond to siloxanes; p-aminobenzoic acid derivatives, such as p-aminobenzoic acid, 2-ethylhexyl p-dimethylaminobenzoate, N-oxypropylenated ethyl p-aminobenzoate, glyceryl p-aminobenzoate; benzophenones such as benzophenone-3, benzophenone-4, 2,2′,4,4′-tetrahydroxy-benzophenone, 2,2′-dihydroxy-4,4′-dimethoxybenzophenone and the like; esters of benzalmalonic acid such as di-(2-ethylhexyl) 4-methoxybenzalmalonate; esters of 2-(4-ethoxy-anilinomethylene)propandioic acid such as 2-(4-ethoxy anilinomethylene) propandioic acid diethyl ester as described in the European Patent Publication EP 0895 776; organosiloxane compounds containing benzmalonate groups as described in the European Patent Publications EP 0358584 B1, EP 0538431 B1 and EP 0709080 A1 such as PARSOL® SLX; drometrizole trisiloxane (Mexoryl XL); imidazole derivatives such as e.g. 2-phenyl benzimidazole sulfonic acid and its salts (PARSOL®HS). Salts of 2-phenyl benzimidazole sulfonic acid are e.g. alkali salts such as sodium- or potassium salts, ammonium salts, morpholine salts, salts of primary, sec. and tert. amines like monoethanolamine salts, diethanolamine salts and the like; salicylate derivatives such as isopropylbenzyl salicylate, benzyl salicylate, butyl salicylate, ethylhexyl salicylate (PARSOL® EHS, Neo Heliopan OS), isooctyl salicylate or homomethyl salicylate (homosalate, PARSOL® HMS, Neo Heliopan HMS) and the like; triazine derivatives such as ethylhexyl triazone (Uvinul T-150), diethylhexyl butamido triazone (Uvasorb HEB) and the like. Encapsulated UV-filters such as encapsulated ethylhexyl methoxycinnamate (Eusolex UV-pearls) or microcapsules loaded with UV-filters as e.g. disclosed in EP 1471995 and the like; Inorganic compounds are pigments such as microparticulated TiO₂, and the like. The term “microparticulated” refers to a particle size from about 5 nm to about 200 nm, particularly from about 15 nm to about 100 nm. The TiO₂ particles may also be coated by metal oxides such as e.g. aluminum or zirconium oxides or by organic coatings such as e.g. polyols, methicone, aluminum stearate, alkyl silane. Such coatings are well known in the art.

Examples of broad spectrum or UV A screening agents i.e. substances having absorption maximums between about 320 nm and 400 nm may be organic or inorganic compounds e.g. dibenzoylmethane derivatives such as 4-tert.-butyl-4′-methoxydibenzoyl-methane (PARSOL® 1789), dimethoxydibenzoylmethane, isopropyldibenzoylmethane and the like; benzotriazole derivatives such as 2,2′-methylene-bis-(6-(2H-benzotriazole-2-yl)-4-(1,1,3,3,-tetramethylbutyl)-phenol (Tinosorb M) and the like; bis-ethylhexyloxyphenol methoxyphenyl triazine (Tinosorb S) and the like; phenylene-1,4-bis-benzimidazolsulfonic acids or salts such as 2,2-(1,4-phenylene)bis-(1H-benzimidazol-4,6-disulfonic acid) (Neoheliopan AP); amino substituted hydroxybenzophenones such as 2-(4-Diethylamino-2-hydroxy-benzoyl)-benzoic acid hexylester (Uvinul A plus) as described in the European Patent Publication EP 1046391; Ionic UV-A filters as described in the International Patent Publication WO2005080341 A1; Pigments such as microparticulated ZnO or TiO₂ and the like. The term “microparticulated” refers to a particle size from about 5 nm to about 200 nm, particularly from about 15 nm to about 100 nm. The particles may also be coated by other metal oxides such as e.g. aluminum or zirconium oxides or by organic coatings such as e.g. polyols, methicone, aluminum stearate, alkyl silane. Such coatings are well known in the art.

As dibenzoylmethane derivatives have limited photostability it may be desirable to photostabilize these UV-A screening agents. Thus, the term “conventional UV-A screening agent” also refers to dibenzoylmethane derivatives such as e.g. PARSOL® 1789 stabilized by, e.g. 3,3-Diphenylacrylate derivatives as described in the European Patent Publications EP 0 514 491 B1 and EP 0 780 119 A1; Benzylidene camphor derivatives as described in the U.S. Pat. No. 5,605,680; Organosiloxanes containing benzmalonate groups as described in the European Patent Publications EP 0358584 B1, EP 0538431 B1 and EP 0709080 A1.

### Antioxidants

Based on the invention all known antioxidants usually formulated into body care and household products can be used. Especially preferred are antioxidants chosen from the group consisting of amino acids (e.g. glycine, histidine, tyrosine, tryptophan) and their derivatives, imidazole (e.g. urocanic acid) and derivatives, peptides such as D,L-carnosine, D-carnosine, L-carnosine and derivatives (e.g. anserine), carotenoids, carotenes (e.g. α-carotene, β-carotene, lycopene) and derivatives, chlorogenic acid and derivatives, lipoic acid and derivatives (e.g. dihydrolipoic acid), aurothioglucose, propylthiouracil and other thiols (e.g. thioredoxine, glutathione, cysteine, cystine, cystamine and its glycosyl-, N-acetyl-, methyl-, ethyl-, propyl-, amyl-, butyl- and lauryl-, palmitoyl-; oleyl-, linoleyl-, cholesteryl- and glycerylester) and the salts thereof, dilaurylthiodipropionate, distearylthiodipropionate, thiodipropionic acid and its derivatives (ester, ether, peptides, lipids, nucleotides, nucleosides and salts) as well as sulfoximine compounds (such as buthioninsulfoximine, homocysteinesulfoximine, buthioninsulfone, penta-, hexa-, heptathioninsulfoximine) in very low compatible doses (e.g. pmol bis μmol/kg), additionally (metal)-chelators (such as α-hydroxyfatty acids, palmic-, phytinic acid, lactoferrin), β-hydroxyacids (such as citric acid, lactic acid, malic acid), huminic acid, gallic acid, gallic extracts, bilirubin, biliverdin, EDTA, EGTA and its derivatives, unsaturated fatty acids and their derivatives (such as γ-linoleic acid, linolic acid, oleic acid), folic acid and its derivatives, ubiquinone and ubiquinol and their derivatives, vitamin C and derivatives (such as ascorbylpalmitate and ascorbyltetraisopalmitate, Mg-ascorbylphosphate, Na-ascorbylphosphate, ascorbyl-acetate), tocopherol and derivates (such as vitamin-E-acetate), mixtures of nat. vitamin E, vitamin A and derivatives (vitamin-A-palmitate and -acetate) as well as coniferylbenzoate, rutinic acid and derivatives, α-glycosylrutin, ferulic acid, furfurylideneglucitol, carnosine, butylhydroxytoluene, butylhydroxyanisole, trihydroxybutyrophenone, urea and its derivatives, mannose and derivatives, zinc and derivatives (e.g. ZnO, ZnSO₄), selen and derivatives (e.g. selenomethionin), stilbenes and derivatives (such as stilbenoxide, trans-stilbenoxide) and suitable derivatives (salts, esters, ethers, sugars, nucleotides, nucleosides, peptides and lipids) of the named active ingredients. One or more preservatives/antioxidants may be present in an amount of at least 0.01 wt. % of the total weight of the composition. Preferably about 0.01 wt. % to about 10 wt. % of the total weight of the composition of the present invention is present. Most preferred, one or more preservatives/antioxidants are present in an amount about 0.1 wt. % to about 1 wt. %.

### Surface Active Ingredients

Typically cosmetic or dermatological compositions also contain surface active ingredients like emulsifiers, solubilizers and the like. An emulsifier enables two or more immiscible components to be combined homogeneously. Moreover, the emulsifier acts to stabilize the composition. Emulsifiers that may be used in the present invention in order to form O/W, W/O, O/W/O or W/O/W emulsions/micro emulsions include sorbitan oleate, sorbitan sesquioleate, sorbitan isostearate, sorbitan trioleate, polyglyceryl-3-diisostearate, polyglycerol esters of oleic/isostearic acid, polyglyceryl-6 hexaricinolate, polyglyceryl-4-oleate, polygylceryl-4 oleate/PEG-8 propylene glycol cocoate, oleamide DEA, TEA myristate, TEA stearate, magnesium stearate, sodium stearate, potassium laurate, potassium ricinoleate, sodium cocoate, sodium tallowate, potassium castorate, sodium oleate, and mixtures thereof. Further exemplary emulsifiers are phosphate esters and the salts thereof such as cetyl phosphate (Amphisol® A), diethanolamine cetyl phosphate (Amphisol® DEA), potassium cetyl phosphate (Amphisol® K), sodium glyceryl oleate phosphate, hydrogenated vegetable glycerides phosphate and mixtures thereof. Furthermore, one or more synthetic polymers may be used as an emulsifier. For example, PVP eicosene copolymer, acrylates/C₁₀₋₃₀ alkyl acrylate crosspolymer, acrylates/steareth-20 methacrylate copolymer, PEG-22/dodecyl glycol copolymer, PEG-45/dodecyl glycol copolymer, and mixtures thereof. Further exemplary emulsifiers are fatty alcohols, e.g. cetearyl alcohol (Lanette O, Cognis Coopearation), cetyl alcohol (Lanette 16, Cognis Cooperation), stearyl alcohol (Lanette 18, Cognis Cooperation), Laneth-5 (Polychol 5, Croda Chemicals), furthermore sucrose and glucose derivatives, e.g. sucrose distearate (Crodesta F-10, Croda Chemicals), Methyl glucose isostearate (Isolan IS, Degussa Care Chemicals), furthermore ethoxylated carboxylic acids or polyethyleneglycol esters and polyethyleneglycol ethers, e.g. steareth-2 (Brij 72, Uniqema), steareth-21 (Brij 721, Uniqema), ceteareth-25 (Cremophor A25, BASF Cooperation), PEG-40 hydrogenated castor oil (Cremophor RH-40, BASF Cooperation), PEG-7 hydrogenated castor oil (Cremophor WO7, BASF Cooperation), PEG-30 Dipolyhydroxystearate (Arlacel P 135, Uniqema), furthermore glyceryl esters and polyglyceryl esters, e.g. polyglyceryl-3-diisostearate (Hostacerin TGI, Clariant Cooperation), polyglyceryl-2 dipolyhydroxystearate (Dehymuls PGPH, Cognis Cooperation), polyglyceryl-3 methylglucose distearate (Tego Care 450, Degussa Care Chemicals). The preferred emulsifiers are cetyl phosphate (Amphisol® A), diethanolamine cetyl phosphate (Amphisol® DEA), potassium cetyl phosphate (Amphisol® K), PVP Eicosene copolymer, acrylates/C₁₀₋₃₀-alkyl acrylate crosspolymer, PEG-20 sorbitan isostearate, sorbitan isostearate, and mixtures thereof. The one or more emulsifiers are present in a total amount of at least 0.01 wt. % of the total weight of the composition. Preferably about 0.01 wt. % to about 20 wt. % of the total weight of the composition of the present invention is used. Most preferred, about 0.1 wt. % to about 10 wt. % of emulsifiers are used.

Typically cosmetic or dermatological compositions may also contain anionic, neutral, amphoteric or cationic tensides.

Exemplary anionic tensides comprise alkylsulfate, alkylethersulfate, alkylsulfonate, alkylarylsulfonate, alkylsuccinate, alkylsulfosuccinate, N-alkoylsarkosinate, acyltaurate, acylisethionate, alkylphosphate, alkyletherphosphate, alkylethercarboxylate, alpha-olefinsulfonate, especially the alkali-und earth alkali salts, e.g. sodium, potassium, magnesium, calcium, as well as ammonium- and triethanol amine-salts. The alkylethersulfate, alkyletherphosphate and alkylethercarboxylate may comprise between 1 to 10 ethylenoxide or propylenoxide units, preferably 1 to 3 ethylenoxide-units per molecule.

Suitable are e.g. sodium laurylsulfate, ammonium lauryl sulfate, sodium laurylethersulfate, ammonium laurylethersulfate, sodium lauroylsarkonisate, sodiumoleylsuccinate, ammonium laurylsulfosuccinate, sodium dodecylbenzolsulfonate, triethanolamidodecylbenzolsulfonate.

Suitable amphoteric tensides are e.g. alkylbetaine, alkylamidopropylbetaine, alkylsulfobetaine, alkylglycinate, alkylcarboxyglycinate, alkylamphoacetate or propionate, alkylamphodiacetate or dipropionate such as cocodimethyl-sulfopropylbetain, laurylbetain, cocamidopropylbetain or sodium cocamphopropionate.

Furthermore, the cosmetic or dermatological compositions may contain the usual cationic tensides such as quaternized ammonium compounds e.g. cetyltrimethylammoniumchlorid or bromide (INCI: cetrimoniumchloride or bromide), hydroxyethylcetyldimonium phosphate (INCI: Quaternium-44), Luviquat® Mono LS (INCI: Cocotrimoniummethosulfate), poly(oxy-1,2-Ethandiyl), (Octadecylnitrilio)tri-2,1-Ethandiyl)tris-(hydroxy)-phosphate (INCI Quaternium-52).

The one or more anionic, neutral, amphoteric or cationic tensides are present in a total amount of at least 0.01 wt. % of the total weight of the composition. Preferably about 0.01 wt. % to about 20 wt. % of the total weight of the composition of the present invention is used. Most preferred, about 0.1 wt. % to about 10 wt. % of one or more tensides are used.

### Oil and Fatty Components

The lipid phase can advantageously be chosen from mineral oils and mineral waxes; oils such as triglycerides of caprinic acid and/or caprylic acid or castor oil; oils or waxes and other natural or synthetic oils, in an preferred embodiment esters of fatty acids with alcohols e.g. isopropanol, propyleneglycol, glycerin or esters of fatty alcohols with carbonic acids or fatty acids; alkylbenzoates; and/or silicone oils.

Exemplary fatty substances which can be incorporated in the oil phase of the emulsion, micro emulsion, oleo gel, hydrodispersion or lipodispersion of the present invention are advantageously chosen from esters of saturated and/or unsaturated, linear or branched alkyl carboxylic acids with 3 to 30 carbon atoms, and saturated and/or unsaturated, linear and/or branched alcohols with 3 to 30 carbon atoms as well as esters of aromatic carboxylic acids and of saturated and/or unsaturated, linear or branched alcohols of 3-30 carbon atoms. Such esters can advantageously be selected from octylpalmitate, octylcocoate, octylisostearate, octyldodecylmyristate, cetearylisononanoate, isopropylmyristate, isopropylpalmitate, isopropylstearate, isopropyloleate, n-butylstearate, n-hexyllaurate, n-decyloleate, isooctylstearate, isononylstearate, isononylisononanoate, 2-ethyl hexylpalmitate, 2-ethylhexyllaurate, 2-hexyldecylstearate, 2-octyldodecylpalmitate, stearylheptanoate, oleyloleate, oleylerucate, erucyloleate, erucylerucate, tridecylstearate, tridecyltrimellitate, as well as synthetic, half-synthetic or natural mixtures of such esters e.g. jojoba oil.

Other fatty components suitable for cosmetic or dermatological compositions of the present invention include polar oils such as lecithins and fatty acid triglycerides, namely triglycerol esters of saturated and/or unsaturated, straight or branched carboxylic acid with 8 to 24 carbon atoms, preferably of 12 to 18 carbon atoms whereas the fatty acid triglycerides are preferably chosen from synthetic, half synthetic or natural oils (e.g. cocoglyceride, olive oil, sun flower oil, soybean oil, peanut oil, rape seed oil, sweet almond oil, palm oil, coconut oil, castor oil, hydrogenated castor oil, wheat oil, grape seed oil, macadamia nut oil and others); apolar oils such as linear and/or branched hydrocarbons and waxes e.g. mineral oils, vaseline (petrolatum); paraffins, squalane and squalene, polyolefins, hydrogenated polyisobutenes and isohexadecanes, favored polyolefins are polydecenes; dialkyl ethers such as dicaprylylether; linear or cyclic silicone oils such as preferably cyclomethicone (octamethylcyclotetrasiloxane; cetyldimethicone, hexamethylcyclotrisiloxane, polydimethylsiloxane, poly(methylphenylsiloxane) and mixtures thereof.

Other fatty components which can advantageously be incorporated in cosmetic or dermatological compositions of the present invention are isoeikosane; neopentylglycoldiheptanoate; propyleneglycoldicaprylate/dicaprate; caprylic/capric/diglycerylsuccinate; butyleneglycol caprylat/caprat; C₁₂₋₁₃-alkyllactate; di-C₁₂₋₁₃-alkyltartrate; triisostearin; dipentaerythrityl hexacaprylat/hexacaprate; propyleneglycolmonoisostearate; tricaprylin; dimethylisosorbid. Especially beneficial is the use of mixtures C₁₂₋₁₅-alkylbenzoate and 2-ethylhexylisostearate, mixtures C₁₂₋₁₅-alkylbenzoate and isotridecylisononanoate as well as mixtures of C₁₂₋₁₅-alkylbenzoate, 2-ethylhexylisostearate and isotridecylisononanoate.

The oily phase of the compositions of the present invention can also contain natural vegetable or animal waxes such as bee wax, china wax, bumblebee wax and other waxes of insects as well as shea butter and cocoa butter.

### Silicone Oils

Suitable silicone oils are e.g. such as dimethylpolysiloxane, diethylpolysiloxane, diphenylpolysiloxane, cyclic siloxanes, poly(methylphenylsiloxanes) as well as amino-, fatty acid-, alcohol-, polyether-, epoxy-, fluoro-, glycoside-, and/or alkyl modified silicone compounds which are liquid or solid at room temperature and mixtures thereof. The number average molecular weight of the dimethicones and poly-(methylphenylsiloxanes) is preferably in the range of 100 to 150000 g/mol. Preferred cyclic siloxanes comprise 4- to 8-membered rings which are for example commercially available as cyclomethicones.

An oil or fatty component is present in an amount of about 1 wt. % to about 50 wt. % of the total weight of the product. The preferred amount of an oil or fatty component is about 2 wt. % to about 25 wt. %, and most preferably about 3 wt. % to about 20 wt.

### Moisturizing Agents

A moisturizing agent may be incorporated into a product of the present invention to maintain hydration or rehydrate the skin. Moisturizers that prevent water from evaporating from the skin by providing a protective coating are called emollients. Additionally an emollient provides a softening or soothing effect on the skin surface and is generally considered safe for topical use. Preferred emollients include mineral oils, lanolin, petrolatum, capric/caprylic triglyceraldehydes, cholesterol, silicones such as dimethicone, cyclomethicone, almond oil, jojoba oil, avocado oil, castor oil, sesame oil, sunflower oil, coconut oil and grape seed oil, cocoa butter, olive oil, aloe extracts, fatty acids such as oleic and stearic, fatty alcohols such as cetyl and hexadecyl (ENJAY), diisopropyl adipate, hydroxybenzoate esters, benzoic acid esters of C₉₋₁₅-alcohols, isononyl iso-nonanoate, ethers such as polyoxypropylene butyl ethers and polyoxypropylene cetyl ethers, and C₁₂₋₁₅-alkyl benzoates, and mixtures thereof. The most preferred emollients are hydroxybenzoate esters, aloe vera, C₁₂₋₁₅-alkyl benzoates, and mixtures thereof. An emollient is present in an amount of about 1 wt. % to about 50 wt. % of the total weight of the product. The preferred amount of emollient is about 2 wt. % to about 25 wt. %, and most preferably about 3 wt. % to about 15 wt. %.

Moisturizers that bind water, thereby retaining it on the skin surface are called humectants. Examples of humectants which can be incorporated into a product of the present invention are glycerin, propylene glycol, polypropylene glycol, polyethylene glycol, lactic acid, sodium lactate, pyrrolidone carboxylic acid, urea, phospholipids, collagen, elastin, ceramides, lecithin, sorbitol, PEG-4, and mixtures thereof. Additional suitable moisturizers are polymeric moisturizers of the family of water soluble and/or swellable/and/or with water gelating polysaccharides such as hyaluronic acid, chitosan and/or a fructose rich polysaccharide which is e.g. available as Fucogel®1000 (CAS-Nr. 178463-23-5) by SOLABIA S. One or more humectants are optionally present at about 0.5 wt. % to about 8 wt. % in a product of the present invention, preferably about 1 wt. % to about 5 wt. %.

The aqueous phase of the products of the present invention can contain the usual cosmetic additives such as alcohols, especially lower alcohols, preferably ethanol and/or isopropanol, low diols or polyols and their ethers, preferably propyleneglycol, glycerin, ethyleneglycol, ethyleneglycol monoethyl- or monobutylether, propyleneglycol monomethyl- or -monoethyl- or -monobutylether, diethyleneglycol monomethyl- or monoethylether and analogue products, polymers, foam stabilizers; electrolytes and especially one or more thickeners.

### Thickeners

Thickeners that may be used in formulations of the present invention to assist in making the consistency of a product suitable include carbomer, siliciumdioxide, magnesium and/or aluminum silicates, lipid thickeners, e.g. cetyl alcohol, cetyl palmitate (Cutina CP, Cognis Cooperation), glyceryl myristate (Estol 3650, Uniqema), microcrystalline wax (A&E Connock), myristyl alcohol (Lanette 14, Cognis Cooperation), myristyl lactate (Crodamol ML, Croda Chemicals), beeswax (A&E Connock), stearic acid (Lipo Chemicals), stearyl alcohol (Lanette 18, Cognis Cooperation), polysaccharides and their derivatives such as xanthan gum (Keltrol, CP Kelco), hydroxypropyl cellulose (Klucel, Hercules Incorporated), Hydroxyethylcellulose (Tylose H, Clariant Corporation), polyacrylamides, self emulsifying polyacrylamide, e.g. Salcare SC 91, Salcare SC 96 (Ciba Specialty Chemicals), Sepigel 305 (Seppic), acrylate crosspolymers, preferably a carbomer, such as Carbopole® of type 980, 981, 1382, 2984, 5984, ETD 2001, ETD 2050, Ultrez 10, Ultrez 21 (Noveon Inc.), alone or mixtures thereof. Thickeners can be present in an amount of about 0.01 wt. % to about 8 wt. % in the product of the present invention, preferably, 0.05 wt. % to about 5 wt. %.

### Neutralizing Agents

Examples of neutralizing agents which may be included in the composition of the present invention to neutralize components such as e.g. an emulsifier or a foam builder/stabilizer include but are not limited to alkali hydroxides such as a sodium and potassium hydroxide; organic bases such as diethanolamine (DEA), triethanolamine (TEA), aminomethyl propanol, and mixtures thereof; amino acids such as arginine and lysine and any combination of any foregoing. The neutralizing agent can be present in an amount of about 0.01 wt. % to about 8 wt. % in the product of the present invention, preferably, 1 wt. % to about 5 wt. %.

### Electrolytes

The addition of electrolytes into the product of the present invention may be necessary to change the behavior of a hydrophobic emulsifier. Thus, the emulsions/microemulsions of this invention may contain preferably electrolytes of one or several salts including anions such as chloride, sulfate, carbonate, borate and aluminate, without being limited thereto. Other suitable electrolytes can be on the basis of organic anions such as, but not limited to, lactate, acetate, benzoate, propionate, tartrate and citrate. As cations preferably ammonium, alkylammonium, alkali- or alkaline earth metals, magnesium-, iron- or zinc-ions are selected. Especially preferred salts are potassium and sodium chloride, magnesium sulfate, zinc sulfate and mixtures thereof. Electrolytes can be present in an amount of about 0.01 wt. % to about 8 wt. % in the product of the present invention.

### Light Stabilizers

The addition of further light stabilizers may be desirable. Such light stabilizers are e.g. known as sterically hindered amine light stabilizer (HALS) which can be of monomeric or polymeric nature. They are for example selected from the group consisting of N,N′-bisformyl-N,N′-bis-(2,2,6,6-tetramethyl-4-piperidinyl)-hexamethylenediamine (Uvinul 4050H), bis-(2,2,6,6-tetramethyl-4-piperidyl)sebacate (Uvinul 4077H), bis-(1,2,2,6,6-pentamethyl-4-piperidyl)-sebacate+methyl-(1,2,2,6,6-pentamethyl-4-piperidyl)-sebacate. (Uvinul 4092H), bis-(2,2,6,6-tetramethylpiperidine-4-yl)-sebacate, bis-(2,2,6,6-tetramethylpiperidine-4-yl)succinate, bis-(1,2,2,6,6-pentamethylpiperidine-4-yl)sebacate, n-butyl-3,5-di-tert-butyl-4-hydroxybenzyl-malonic acid bis-(1,2,2,6,6-pentamethylpiperidyl)ester, the condensate of 1-hydroxyethyl-2,2,6,6-tetramethyl-4-hydroxypiperidine and succinic acid, the condensate of N,N′-bis-(2,2,6,6-tetramethyl-4-piperidyl)hexamethylenediamine and 4-tert-octylamino-2,6-dichloro-1,3,5-s-triazine, tris-(2,2,6,6-tetramethyl-4-piperidyl)nitrilotriacetate, tetrakis-(2,2,6,6-tetra-methyl-4-piperidyl)-1,2,3,4-butanetetranoate, 1,1′-(1,2-ethanediyl)-bis(3,3,5,5-tetramethyl-piperazinone), 4-benzoyl-2,2,6,6-tetramethylpiperidine, 4-stearyloxy-2,2,6,6-tetramethylpiperidine, bis-(1,2,2,6,6-pentamethylpiperidyl)-2-n-butyl-2-(2-hydroxy-3,5-di-tert-butylbenzyl)malonate, 3-n-octyl-7,7,9,9-tetramethyl-1,3,8-triazaspiro[4.5]decan-2,4-dione, the condensate of N,N-bis-(2,2,6,6-tetramethyl-4-piperidyl)hexamethylenediamine and 4-morpholino-2,6-dichloro-1,3,5-triazine, the condensate of 2-chloro-4,6-di(4-n-butylamino-2,2,6,6-tetra-methylpiperidyl)-1,3,5-triazine and 1,2-bis-(3-aminopropylamino) ethane, the condensate of 2-chloro-4,6-di(4-n-butylamino-1,2,2,6,6-pentamethylpiperidyl)-1,3,5-triazine and 1,2-bis-(3-aminopropylamino)ethane, 8-acetyl-3-dodecyl-7,7,9,9-tetramethyl-1,3,8-triazaspiro[4.5]-decane-2,4-dione, 3-dodecyl-1-(2,2,6,6-tetramethyl-4-piperidyl)pyrrolidin-2,5-dione, 3-dodecyl-1-(1,2,2,6,6-pentamethyl-14-piperidyl)-pyrrolidine-2,5-dione, a mixture of 4-hexadecyloxy- and 4-stearyloxy-2,2,6,6-tetramethylpiperidine, the condensate of N,N′-bis-(2,2,6,6-tetramethyl-4-piperidyl)hexamethylenediamine and 4-cyclohexylamino-2,6-dichloro-1,3,5-triazine, the condensate of 1,2-bis(3-aminopropylamino)ethane and 2,4,6-trichloro-1,3,5-triazine and 4-butylamino-2,2,6,6-tetramethylpiperidine (CAS reg. No. [136504-96-6]); (2,2,6,6-tetramethyl-4-piperidyl)-n-dodecylsuccinimide, (1,2,2,6,6-pentamethyl-4-piperidyl)-n-dodecylsuccinimide, 2-undecyl-7,7,9,9-tetramethyl-1-oxa-3,8-diaza-4-oxo-spiro[4,5]decane, the reaction product of 7,7,9,9-tetramethyl-2-cycloundecyl-1-oxa-3,8-diaza-4-oxospiro[4,5]decane and epichlorohydrin without being limited thereto.

### Film Formers

Customary film formers include, for example, chitosan, microcrystalline chitosan, quaternized chitosan, polyvinylpyrrolidone, vinylpyrrolidone/vinyl acetate copolymers, polymers of quaternary cellulose derivatives containing a high proportion of acrylic acid, collagen, hyaluronic acid and salts thereof and similar compounds.

### Polymers

The products according to the invention may comprise additional cosmetically and dermatologically acceptable polymers in order to established the desired properties. For this purpose all anionic, cationic, amphoteric or neutral polymers may be used.

Examples of cationic polymers are Polyquaternium (INCI), e.g. copolymers of vinylpyrrolidone/N-vinylimidazolium salts (Luviquat®FC, Luviquat® HM, Luviquat®MS, Luviquat®Ultracare), copolymers of N-vinylpyrrolidone/dimethylamino-ethylmethacrylate, quaternized with diethylsulfate (Luviquat® PQ 11, INCI: Polyquaternium-11), copolymers of N-vinylcaprolactam/N-vinyl-pyrrolidone/N-vinylimidazolium salts (Luviquat®Hold; INCI: Polyquaternium-46); cationic derivatives of cellulose (Polyquaternium-4 und -10), acrylamidocopolymers (Polyquaternium-7), Chitosan, cationic starch derivatives (INCI: starch hydroxypropyltrimonium chloride, corn starch modified), cationic guar derivates (INCI: hydroxypropyl guar hydroxypropyltrimonium chloride), cationic sun flower seed derivatives (INCI: sun flower seed amidopropyl hydroxyethyldimonium chloride), copolymers of acrylic acid, acrylamide and methacrylamidopropyltrimoniumchlorid (INCI: Polyquaternium-53), Polyquaternium-32, Polyquaternium-28 without being limited thereto. Suitable cationic quaternized polymers are furthermore Merquat® (polymers on the basis of dimethyldiallyl ammoniumchlorid), Gafquat® (quaternary polymers formed by reacting polyvinylpyrrolidone with quaternary ammonium compounds); Polymer JR (hydroxyethylcellulose with cationic groups), and cationic polymers on plant basis such as guar polymers, commercially available as Jaguar® grades of Rhodia.

Examples of neutral polymers are polyvinylpyrrolidone, copolymers of N-vinylpyrrolidone and vinylacetate and/or vinylpropionate, polysiloxane, polyvinylcaprolactam and other copolymers of N-vinylpyrrolidone, copolymers of N-vinylpyrrolidone and alkylacrylate or methacrylate monomers with C1-C18 alkyl chains, copolymers of polyvinylalcohol and polyalkylenglycole such as Kollicoats® IR (BASF) or copolymers of other vinyl monomers to polyalkylenglycol, polysiloxane, polyvinylcaprolactam and copolymers with N-Vinylpyrrolidone, polyethylenimine and the salts thereof, polyvinylamine and the salts thereof, cellulose derivatives, chitosan, polyasparaginic acid salts and derivatives thereof, polyethylenimine and the salts thereof, polyvinylamine and the salts thereof such as Luviflex® Swing (partly hydrolysed copolymer of polyvinylacetate and polyethylenglycol by BASF).

Suitable polymers are also non-ionic, water soluble respectively water dispersible polymers or oligomers such as polyvinylcaprolactam, e.g. Luviskol® Plus (BASF), or polyvinylpyrrolidone and copolymers with e.g. vinylesters such as vinylacetate e.g. Luviskol® VA 37 (BASF); polyamide e.g. on the basis of itaconic acid and aliphatic diamines as e.g. described in DE-A-43 33 23.

Additional suitable polymers are nonionic, siloxane-containing, water-soluble or -dispersible polymers, e.g. polyether siloxanes, such as Tegopren® (Goldschmidt) or Belsil® (Wacker).

### Preservatives

Examples of preservatives include Methyl-, Ethyl-, Propyl-, Butylparabens, Benzalkonium chloride, 2-Bromo-2-nitro-propane-1,3-diol, Dehydroacetic acid, Diazolidinyl Urea, 2-Dichlorobenzyl alcohol, DMDM hydantoin, Formaldehyde solution, Methyldibromoglutaronitrile, Phenoxyethanol, Sodium Hydroxymethylglycinate, Imidazolidinyl Urea, Triclosan and further substance classes listed in the following reference: K. F. De Polo-A short textbook of cosmetology, Chapter 7, Table 7-2,7-3, 7-4 and 7-5, p 210-219.

### Scents and Fragrances

The cosmetic or dermatological compositions according to the invention may contain scents and fragrances comprising at least one, preferably numerous odorant ingredients of natural and/or synthetic origin. The range of the natural odorants includes, in addition to readily volatile, also moderately and only slightly volatile components. The synthetic odorants embrace representatives from practically all classes of odorant substances.

The following list comprises examples of known odorants without being limited thereto: natural products such as tree moss absolute, basil oil, tropical fruit oils (such as bergamot oil, mandarin oil, etc.), mastix absolute, myrtle oil, palmarosa oil, patchouli oil, petitgrain oil, wormwood oil, lavender oil, rose oil, jasmine oil, ylang-ylang oil, etc.; alcohols: farnesol, geraniol, linalool, nerol, phenylethyl alcohol, rhodinol, cinnamic alcohol, (Z)-hex-3-en-1-ol, menthol, a-terpineol, etc.; aldehydes such as citral, alpha-hexyl cinnamaldehyde, Lilial, methylionone, verbenone, nootkatone, geranylacetone, etc.; esters such as allyl phenoxyacetate, benzyl salicylate, cinnamyl propionate, citronellyl acetate, decyl acetate, dimethylbenzylcarbinyl acetate, dimethylbenzylcarbinyl butyrate, ethyl acetoacetate, cis-3-hexenyl isobutyrate, cis-3-hexenyl salicylate, linalyl acetate, methyl dihydrojasmonate, styralyl propionate, vetiveryl acetate, benzyl acetate, geranyl acetate, etc.; lactones such as gamma-undecalactone, delta-decalactone, pentadecanolide, 12-oxahexadecanolide, etc.; acetals such as Viridine (phenylacetaldehyde dimethylacetal), etc.; and other components often used in perfumery such as indole, p-mentha-8-thiol-3-one, methyleugenol, eugenol, anethol, etc.

### Active Ingredients

The cosmetic or dermatological composition of the present invention may further comprise a safe and effective amount of additional skin active agents. The skin active agents included herein are e.g. skin-lightening agents, tanning prevention agents, agents for the treatment of hyperpigmentation, agents for the prevention or reduction of acne, vitamins, emollients, non-steroidal anti-inflammatory agent, topical anaesthetics, antiseptics, antimicrobial (e.g. bacteria-inhibiting) and anti-fungal actives, skin soothing agents, skin barrier repair agents, anti-wrinkle agents, anti-skin atrophy actives, lipids, sebum inhibitors, skin sensates, protease inhibitors, skin tightening agents, skin anti-cellulites agents, anti-itch agents, hair grow inhibitors, desquamation enzyme enhancers, anti-glycation agents, chelators and sequestrants, opacifiers, radical scavengers, desquamatory actives, anti-acne actives, anti-oxidants, and mixtures thereof.

When included, the present cosmetic or dermatological composition comprise at least 0.001 wt. % of the skin active ingredient. Generally, an amount of about 0.001 wt. % to about 30 wt. %, preferably from about 0.001 wt. % to about 10 wt. % of an additional skin active agent is used.

The type and amount of the skin active agents are selected so that the inclusion of a specific agent does not affect the stability of the cosmetic or dermatological composition.

Additional skin-lightening can be added to the cosmetic or dermatological compositions of the invention if an additional increase of the skin whitening efficacy is desired. The use of combinations of skin lightening agents may be advantageous in that they may provide skin lightening benefit through different mechanisms. Preferably, the additional skin lightening agents is selected from bis-pantoyl-cystamine, arbutin and alpha-arbutin, deoxyarbutin, undecylenoyl phenyl alanine (for example, SEPIWHITE MSH available from Seppic), octadecenedioic acid (for example ARLATONE DIOIC DCA available from Uniquema), oenothera biennis sead extract, and pyrus malus (apple) fruit extract, Melfade (available from Pentapharm), MELAWHITE (available from Pentapharm), Melanostatine DM (available from Laboratories Seporga), FADEOUT (available from Pentapharm), GATULINE WHITENING (available from Gattlefosse), kojic acid, kojic dipalmitate, lactic acid, vitamin B₃ compounds, azelaic acid, gallic acid and its derivatives, hydroquinone, mulberry extract, lemon extract, phyllanthus emblica fruit extract (available as Emblica™), leucocyte extract, bearberry extract, licorice extract and mixtures thereof. Examples of additional, other skin lightening agents, which may be present in the compositions of the present invention are especially those disclosed in WO 2004/062635, WO 2004/037213, and DE 102 38 449.

Preferable additional skin lightening agents which may be used in the cosmetic or dermatological compositions according to the present invention are

Kojic acid or derivatives thereof, which may be present in the compositions of the present invention in an amount from about 0.05 wt.-% to about 5 wt.-%;

Arbutin or derivatives thereof which may be present in compositions of the present invention in an amount from about 0.05 wt.-% to about 5 wt.-%;

Hydroquinone or derivatives thereof which may be present in the compositions of the present invention in an amount from about 0.05 wt.-% to about 2 wt.-%;

Phyllanthus Emblica fruit extract (trade name: Emblica™), which may be present in the compositions of the present invention in an amount from 0.05 wt.-% to about 3 wt.-%;

Leucocyte extract, which may be present in the compositions of the present invention in an amount from 0.05 wt.-% to about 3 wt.-%;

Bearberry extract, which may be present in the compositions of the present invention in an amount from 0.05 wt.-% to about 3 wt.-%;

Licorice extract, which may be present in the skin care compositions of the present invention in an amount from 0.05 wt.-% to about 3 wt.-%; and

Mulberry extract, which may be present in the skin care compositions of the present invention in an amount from 0.05 wt.-% to about 3 wt.-%.

Examples of active ingredients are e.g. glycerol, urea, guanidine (e.g., amino guanidine); vitamins and derivatives thereof such as vitamin A (e.g., retinoid derivatives such as retinal, retinyl palmitate or retinyl propionate), vitamin E (e.g., tocopherol acetate), vitamin B₃ (e.g., niacinamide) and vitamin B₅ (e.g., panthenol), vitamin B₆, vitamin B₁₂, vitamin K, vitamin D, folic acid, biotin and the like and mixtures thereof, wax-based synthetic peptides (e.g., octyl palmitate and tribehenin and sorbitan isostearate and palmitoyl-oligopeptide), amino acids, oligopeptides, and bioactive peptides (e.g., Matrixyl™ [pentapeptide derivative]), anti-acne medicaments (e.g. resorcinol); antioxidants (e.g., phytosterols, lipoic acid); flavonoids or polyphenols (e.g., isoflavones, phytoestrogens); skin soothing and healing agents such as aloe vera extract, allantoin and the like; agents suitable for aesthetic purposes such as essential oils or aromatic compounds (e.g., clove oil, menthol, camphor, eucalyptus oil, and eugenol), hydroxy acids, farnesol, antifungal actives such as bisabolol, alkyldiols such as 1,2-pentanediol, hexanediol or 1,2-octanediol, panthenol, phytol, phytanetriol, ceramides and pseudoceramides, protein hydrolysates, AHA acids, polyunsaturated fatty acids, plant extracts like kinetin, DNA or RNA and their fragmentation products or carbohydrates, conjugated fatty acids, carnitin, carnosine, biochinonen, phytofluen, phytoen, and their corresponding derivatives, carotinoid derivatives such as beta carotene, lycopene or asthaxanthene.

A vitamin E derivative for use in the present invention is tocopheryl acetate. Tocopheryl acetate may be present in the skin care products in an amount from about 0.05 wt.-% to about 5 wt.-%. Another vitamin E derivative of interest is tocopheryl linoleate. Tocopheryl linoleate may be present in the skin care composition in an amount from about 0.05 wt.-% to about 5 wt.-%.

Examples of vitamins from the B complex for use in the present invention are vitamin B₃, B₆ and biotin. Vitamin B₃ may be present in the skin care products in an amount from about 0.01 wt.-% to about 1.00 wt.-%. Vitamin B₆ may be present in the skin care products in an amount from about 0.01 wt-% to about 5.00 wt.-%. Biotin may be present in the skin care products in an amount from about 0.001 wt.-% to about 0.5 wt.-Panthenol may be present in the skin care products in an amount from about 0.05 wt.-% to about 5.00 wt.-%. Phytantriol may be present in the skin care products in an amount from about 0.01 wt.-% to about 5 wt.-%. Bisabolol may be present in the skin care products in an amount from about 0.05 wt.-% to about 5.00 wt.-%.

Examples of deodorizing active ingredients which come into consideration are antiperspirants such as aluminum chlorohydrates, aluminum hydroxyacetates and acidic aluminum/zirconium salts. Esterase inhibitors may be added as further deodorizing active ingredients. Such inhibitors are preferably trialkyl citrates, such as trimethyl citrate, tripropyl citrate, triisopropyl citrate, tributyl citrate and especially triethyl citrate (Hydagen CAT, Henkel), which inhibit enzyme activity and hence reduce odor formation. Further substances that come into consideration as esterase inhibitors are sterol sulfates or phosphates, for example lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester and hydroxycarboxylic acids and esters thereof, for example citric acid, malic acid, tartaric acid or tartaric acid diethyl ester. Antibacterial active ingredients that influence the germ flora and kill or inhibit the growth of sweat-decomposing bacteria can likewise be present in the preparations (especially in stick preparations). Other antibacterials which could be present are chitosan, phenoxyethanol and chlorhexidinegluconate-5-chloro-2-(2,4-dichlorophenoxy)-phenol (Triclosan, Irgasan, Ciba Specialty Chemicals Inc.).

Typical examples of bacteria-inhibiting agents are preservatives that have a specific action against gram-positive bacteria, such as 2,4,4′-trichloro-2′-hydroxydiphenyl ether, chlorhexidine (1,6-di-(4-chlorophenyl-biguanido) hexane) or TCC (3,4,4′-trichlorocarbanilide). A large number of aromatic substances and ethereal oils also have antimicrobial properties. Typical examples are the active ingredients eugenol, menthol and thymol in clove oil, mint oil and thyme oil. A natural deodorizing agent of interest is the terpene alcohol farnesol (3,7,11-tri-methyl-2,6,10-dodecatrien-1-ol), which is present in lime blossom oil. Glycerolmonolaurate has also proved to be a bacteriostatic agent. The amount of the additional bacteria-inhibiting agents present is usually from 0.1 to 2 wt. %, based on the solids content of the preparations.

### Insect Repellents

Examples of insect repellents which can be used in products according to the invention are for example N,N-diethyl-m-toluamide, 1,2-pentanediol or insect repellant 3535.

According to the invention the active ingredients incorporated into cosmetic or dermatological compositions according to the invention can be used as such or in an encapsulated form, for example in a liposomal form. Liposomes are preferably formed with lecithins with or without addition of sterols or phytosterols. The encapsulation of the active ingredients can be alone or together with other active ingredients.

Regarding the kind of the topical cosmetic and pharmaceutical composition and the preparation of the topical cosmetic and dermatological preparations as well as for further suitable additives, it can be referred to the pertinent literature, e.g. to Novak G.A., Die kosmetischen Präparate—Band 2, Die kosmetischen Präparate—Rezeptur, Rohstoffe, wissenschaftliche Grundlagen (Verlag für Chem. Industrie H. Ziolkowski K G, Augsburg).

The invention is illustrated further by the Examples

### EXAMPLE 1

A water-in-oil cream for skin lightening was prepared from the components listed below:

| INGREDIENTS | wt. % |
|---|---|
| Aqua | ad 100 |
| Cremophor WO-7 (PEG-7 Hydrogenated Castor Oi)l | 6.0 |
| Myritol 318 (Caprylic/Capric Triglyceride) | 5.0 |
| Mineral Oil | 5.0 |
| Propylene Glycol | 5.0 |
| Jojoba Oil | 5.0 |
| Stay-C ® 50 (Trisodium Ascorbyl Phosphate) | 5.0 |
| Paracera M (microcrystalline wax) | 2.0 |
| Elfacos ST-9 (PEG-45/Dodecyl Glycol Copolymer) | 2.0 |
| Phytantriol | 2.0 |
| Salicylic Acid | 1.5 |
| Disodium EDTA | 0.1 |
| Butylated Hydroxytoluene | 0.05 |

Procedure: Trisodium Ascorbyl Phosphate (Stay-C® 50) was neutralized using to pH 6.0 using salicylic acid. All all oil soluble ingredient are molten together at 85° C. Then add the water phase (it doesn't matter if hot or cold) and stir carefully until a homogeneous cream has been formed. When the emulsion is cool down to 30° C. and homogenise again.

### EXAMPLE 2

The efficacy of salicylic acid in enhancing skin penetration of trisodium ascorbyl phosphate was determined by an in vitro techniques using a Franz-type diffusion cell system. The single glass diffusion cell has an exposed membrane area of 5.0 cm² and a receptor volume of 10 ml.

Human skin samples were obtained from surgery or post mortem and were used at a split thickness of 300 μm. The skin samples from different donors were placed in the diffusion cells and membrane integrity was determined by using the standard method with tritiated water. Cells were assigned such that data could be obtained from intact membranes. The receptor chambers of the cells, each containing a small magnetic stirrer bar, were filled with a recorded volume of receptor fluid (0.9% sodium chloride solution in water) and placed in a water bath maintained at a normal skin temperature of 32±1° C. This receptor fluid ensures that the test substance can freely partition into the receptor fluid from the skin membrane and never reaches a concentration that would limit its diffusion. The formulation of Example 1 was applied undiluted to the skin membranes at a mean dose rate of 48.4 mg/cm² that corresponds to 2541.95 μg trisodium ascorbyl phosphate /cm². Since the test material was a cream, the doses were applied to the skin and spread over the surface using small metal rods. The skin was left unclouded for the duration of the exposure period (24 h).

For calculation of the mass balance the donor chamber was carefully removed, washed with 1% shower gel solution and a sample of the washing fluid was analyzed for trisodium ascorbyl phosphate by HPLC at the end of the experiment (24 h). The epidermal surfaces of the skin was washed by gently swabbing the application site with a series of 3 natural sponges pre-wetted with 1% shower gel solution and with a further 3 dry sponges. To assess penetration through the stratum corneum, successive layers of the stratum corneum were removed by the repeated application of adhesive tape (Scotch 3M Magic Tape, 1.25 cm wide), to a maximum of 10 strips and solved in 1% shower gel solution. The strips were fractionated and analysed as strip 1+2 and strip 3-10. The remaining skin was carefully removed from the receptor chamber. The epidermis was separated from the dermis. The remaining epidermal skin and the dermis were extracted separately in liquid nitrogen and solved in water for HPLC analysis.

### Analytical Method: High Performance Liquid Chromatography

The formulation and skin fraction samples were injected into the HPLC system using DAD detection with the following conditions:

| | |
|---|---|
| HPLC system | Agilent 1100 Series |
| Software | Agilent ChemStation Plus Family LC-MS^{3D} 8.03 |
| | (Windows NT) |
| Column | XTerra RP18, 150 × 3.0 mm, 3.5 μm |
| Mobile Phase | 5 mM KH2PO4, pH 2.5/Methanol (95/5, v/v), |
| | isocratic |
| Flow | 0.4 ml/min |
| Injection volume | 5 μl |
| Detection wavelength | 240 nm |
| Retention time | approx. 4.1 min |
| Analysis time | 7 min |
| Limit of Quantification | 2.5 μg/ml |

The % of penetrated trisodium ascorbyl phosphate was determined with a validated computer program using the following equation:

$$\% penetrated = \frac{Mean amount penetrated ( μg \text{/} {cm}^{2} )}{Mean amount applied ( μg \text{/} {cm}^{2} )} \times 100$$

Mean values were calculated from a total of 16 cells comparing an W/O formulation as mentioned in example 1 containing trisodium ascorbyl phosphate and salicylic acid (Sample A) with a placebo formulation wherein salicylic acid was replaced by citric acid (Sample B).

### Results:

| | % trisodium ascorbyl phosphate penetrated into the | | | |
|---|---|---|---|---|
| | | | | dermis and |
| | stratum corneum | stratum corneum | epi- | receptor |
| | (strips 1-2) | (strips 3-10) | dermis | fluid |
| Sample A | 0.5 | 0.4 | 0.6 | 0.1 |
| Sample B | 0.4 | 0.3 | 0.1 | 0.0 |

The total mean amount of trisodium ascorbyl phosphate penetrated (equals the sum of amount penetrated into the epidermis and dermis and receptor fluid) consequently was 0.7% from a W/O formulation containing 1.5% salicylic acid and 5% trisodium ascorbyl phosphate (molar ratio of salicylic acid (MW 138.12) to trisodium ascorbyl phosphate (STAY-C®50, MW 358.08) of about 0.77) and only 0.1% from the W/O placebo formulation. In conclusion these data suggest that the human dermal absorption of trisodium ascorbyl phosphate from a W/O formulation containing trisodium ascorbyl phosphate plus salicylic acid is significantly higher (more than seven times) compared to the same formulation containing trisodium ascorbyl phosphate alone (placebo).

### EXAMPLE 3

### Anti Pimple Skin-Tonic with Stay-C 50

| Phase | INCI Nomenclature | wt. % |
|---|---|---|
| 1 | Sodium Ascorbyl Phosphate | 5.00 |
| | Alcohol | 15.00 |
| | Glyerin | 3.00 |
| | Aqua (e.g. deionized water) | Ad 100 |
| | Disodium EDTA | 0.10 |
| 2 | Salicylic Acid | 1.50 |

Procedure: Add all ingredients of part 1 and mix intensively until a homogeneous solution is obtained. Adjust the pH to 6.5 with part 2.

### EXAMPLE 4

### Anti-Acne Treatment with Stay-C 50

| Phase | INCI Nomenclature | wt. % |
|---|---|---|
| 1 | Glyceryl Myristate | 1.50 |
| | Cetyl Alcohol | 1.50 |
| | C12-15 Alkyl Benzoate | 4.00 |
| | Phenoxyethanol & Methylparaben & Ethylparaben & | 0.80 |
| | Butylparaben & Propylparaben & Isobutylparaben | |
| | Isononyl Isononanoate | 2.00 |
| | Steareth-2 | 1.50 |
| | Steareth-21 | 1.50 |
| 2 | Butylene Glycol | 2.00 |
| | Glycerin | 3.00 |
| | Disodium EDTA | 0.10 |
| | Xanthan Gum | 0.30 |
| | Arcylates/C10-30 Alkyl Acrylate Crosspolymer | 0.25 |
| | Aqua (e.g. deionized water) | Ad 100 |
| 3 | Aqua (e.g. deionized water) | 10.00 |
| | Sodium Ascorbyl Phosphate | 3.00 |
| | Sodium Metabisulfite | 0.05 |
| 4 | Salicylic Acid | 0.60 |

Procedure: Heat part 1 up to 85° C.; and heat also part 2 up to 85° C. When both have the same temperature add part 2 to part 1 while homogenizing intensively. Cool down the product to 35° C. while stirring. Now add part 3 and 4 homogenize intensively again. It is generally recommended to use vacuum while producing the emulsion.

### EXAMPLE 5

### Skin Lightening Cream with STAY-C50

| Phase | INCI Nomenclature | wt. % |
|---|---|---|
| 1 | Glyceryl Myristate | 2.50 |
| | Cetyl Alcohol | 2.50 |
| | Ethylhexyl Methoxycinnamate | 5.00 |
| | Butyl Methoxydibenzoylmethane | 2.00 |
| | Octocrylene | 1.70 |
| | Prunus Amygdalus Dulcis (Sweet Almond) Oil | 2.00 |
| | C12-15 Alkyl Benzoate | 4.00 |
| | BHT | 0.05 |
| | Dimethicone | 0.50 |
| | Tocopheryl Acetate | 1.00 |
| | Phenoxyethanol & Methylparaben & Ethylparaben & | 0.80 |
| | Butylparaben & Propylparaben & Isobutylparaben | |
| | Steareth-2 | 2.00 |
| | Steareth-21 | 2.00 |
| | Cyclomethicone | 4.00 |
| 2 | Aqua (e.g. deionized water) | Ad 100 |
| | Butylene Glycol | 2.00 |
| | Glycerin | 3.00 |
| | Disodium EDTA | 0.10 |
| | Xanthan Gum | 0.20 |
| | Acrylates/C10-30 Alkyl Acrylate Crosspolymer | 0.25 |
| 3 | Salicylic Acid | 1.20 |
| | Sodium Ascorbyl Phosphate | 3.00 |

Procedure: Heat part 1 and part 2 to 85° C. When both have the same temperature add part 2 into part 1 under agitation. Add the solution of part 3 under agitation and homogenization. Cool down the emulsion to room temperature while stirring.

### EXAMPLE 6

### Anti Acne Skin Tonic

| INCI Nomenclature | wt. % |
|---|---|
| Alcohol | 15.00 |
| Sodium Ascorbyl Phosphate | 1.50 |
| Salicylic Acid | 0.4 |
| Glycerin | 3.00 |
| Propylene Glycol & Diazolidinyl Urea & | 0.50 |
| Methylparaben & Propylparaben | |
| Aqua (e.g. deionized water) | Ad 100 |
| Sodium Metabisulfite | 0.01 |
| PEG-40 Hydrogenated Castor Oil | 1.50 |
| Panthenol | 0.50 |
| Propylene Glycol & Menthol & Water & | 0.30 |
| Rosmarinus Officinalis (Rosemary) | |
| Leaf Extract & Ethoxydiglycol | |
| Perfume | 0.10 |

Procedure: mix all ingredients together until a clear solution has been obtained

### EXAMPLE 7

### Anti Acne Gel

| INCI Nomenclature | wt. % |
|---|---|
| Sodium Ascorbyl Phosphate | 1.50 |
| Salicylic Acid | 0.6 |
| Glycerin | 3.00 |
| Acrylates/C10-30 Alkyl Acrylate Crosspolymer | 1.00 |
| Xanthan Gum | 0.50 |
| Sodium Hydroxide | 0.40 |
| Propylene Glycol & Diazolidinyl Urea & | 0.50 |
| Methylparaben & Propylparaben | |
| Aqua (e.g. deionized water) | Ad 100 |
| Sodium Metabisulfite | 0.01 |
| PEG-40 Hydrogenated Castor Oil | 1.50 |
| Panthenol | 0.50 |
| Propylene Glycol & Menthol & Water & | 0.30 |
| Rosmarinus Officinalis (Rosemary) | |
| Leaf Extract & Ethoxydiglycol | |
| Perfume | 0.10 |

Procedure: Dispers Carbopol and Xanthan in Water and homogenize intensively. Add the rest of the ingredients under agitation until everything is solved. Remove the air bubbles under vacuum

| INCI Nomenclature | wt. % |
|---|---|
| Sodium Ascorbyl Phosphate | 1.50 |
| Salicylic Acid | 0.3 |
| Glycerin | 3.00 |
| Sodium Laureth Sulfate | 35.00 |
| Xanthan Gum | 0.50 |
| Cocamidopropyl Betaine | 5.00 |
| PEG-7 Glyceryl Cocoate | 3.00 |
| Disodium EDTA | 0.10 |
| Propylene Glycol & Diazolidinyl Urea & | 0.50 |
| Methylparaben & Propylparaben | |
| Aqua (e.g. deionized water) | Ad 100 |
| Sodium Metabisulfite | 0.01 |
| Sodium Chloride | 1.50 |
| PEG-40 Hydrogenated Castor Oil | 1.50 |
| Panthenol | 0.50 |
| Propylene Glycol & Menthol & Water & | 1.50 |
| Rosmarinus Officinalis (Rosemary) | |
| Leaf Extract & Ethoxydiglycol | |
| Perfume | 0.10 |

Procedure: Add all ingredients without the water and mix intensively until a homogeneous solution is obtained. Add the water under slow agitation and wait until the foam has disappeared. Add Sodium Chloride to adjust the viscosity.

1. A cosmetic or dermatological composition comprising
(a) 0.01 to 8 wt. % of salicylic acid, optionally substituted salicylic acid and/or derivatives thereof;
(b) 0.01 to 20 wt. % of ascorbic acid and/or derivatives thereof and
(c) a cosmetically acceptable carrier.

2. The cosmetic or dermatological compositions as in claim 1 wherein the molar ratio of the salicylic acid, optionally substituted salicylic acid and/or derivatives thereof to the ascorbic acid and/or derivatives thereof is in the range of 0.1:5 to 5:1, preferably in the range of 0.5:2 to 2:0.5, most preferred in the range of about 0.7:1.2 to 1.2:0.7.

3. The cosmetic or dermatological composition as in claim 1 wherein the ascorbic acid and/or derivatives thereof are selected from sodium or magnesium, or sodium magnesium or sodium calcium ascorbyl phosphate.

4. The cosmetic or dermatological composition as in claim 1, wherein the ascorbyl phosphate is trisodium-L-ascorbyl-2-monophosphate.

5. The cosmetic or dermatological composition as in claim 1, wherein the salicylic acid, optionally substituted salicylic acid and/or derivatives thereof are selected from salicylic acid, sodium salicylate, potassium salicylate and/or magnesium salicylate.

6. Use of cosmetic or dermatological compositions of claim 1 for the control of melanin formation, for enhancing the collagen deposition and for an efficient antioxidant protection.

7. Use of an effective amount of a cosmetic or dermatological compositions of claim 1 for skin lightening and/or for the prevention, reduction or treatment of acne and/or skin ageing.

8. Method of lightening the skin comprising the step of applying an effective amount of the composition of claim 1 to the skin of a subject in need of such a treatment.

9. Method of providing anti-ageing benefit to the skin comprising the step of applying an effective amount of the composition of claim 1 to the skin of a subject in need of such a treatment.

10. Method of prevention, reduction or treatment of acne comprising the step of applying an effective amount of the composition of claim 1 to the skin of a subject in need of such a treatment.

11. Use of salicylic acid, optionally substituted salicylic acid and/or derivatives thereof in cosmetic or dermatological compositions for enhancing the penetration of ascorbic acid and/or derivatives thereof into the human skin.

12. Method of enhancing the efficacy of ascorbic acid and/or derivatives thereof which comprises adding to a composition containing an ascorbic acid and/or derivatives thereof an effective amount of salicylic acid, optionally substituted salicylic acid and/or derivatives thereof.
